(19) United States
(12) Patent Application Publication
(10) Pub. No.: US 2025/0283865 A1
(43) Pub. Date: Sep. 11, 2025

(54) METHODS AND SYSTEMS FOR ESTIMATING AN AMOUNT OF A SEQUESTERED CARBON CONSTITUENT IN TOP AND SUB SOIL

(71) Applicant: QuantumPro, Inc., Houston, TX (US)

(72) Inventor: Talgat Shokanov, Houston, TX (US)

(21) Appl. No.: 18/599,007

(22) Filed: Mar. 7, 2024

(51) Int. Cl. G01N 33/24; G01N 1/08

(52) U.S. Cl. CPC G01N 33/24; G01N 1/08

(57) ABSTRACT

A method of estimating an amount of a sequestered carbon-based constituent in a volume of soil that includes taking a core soil sample of a sample volume of soil proximate at and below a root system associated with carbon soil accumulation from a biomass. The core sample is taken at a depth extending from an earthen surface, the depth having a depth range. The core sample is tested to provide a data set.

### BACKGROUND

### Field of the Disclosure

This disclosure generally relates to using innovative and unique methods and systems of taking a core sample of soil, testing, and analyzing the core sample to estimate or validate an amount of a sequestered and deposited carbon constituent, such as carbon dioxide, at various depths.

### Background of the Disclosure

A hydrocarbon-based economy continues to be the dominant force in the modern world. As such, locating, producing, and burning fossil fuels (hydrocarbons) continues to be part of everyday life. These human activities are believed to contribute to significant alteration of natural environments in all parts of the world and to the vast amounts of continued greenhouse gas emissions (such as carbon dioxide or ‘CO2’) in the atmosphere.

In recent years, reducing the amount of CO2 in the atmosphere has taken on considerable interest. Platforms and legislation that encourage or promote environmentally sound governance (ESG) are driving companies to seek net zero emission goals. Engineered and technical solutions are one possibility, but these are often in the form of high capital expenditure and require significant lead time (such as decades), and do not provide any sense of immediate realization on investment. One current example is removing CO2 from the air mechanically, and injecting it into a geological formation. This high-cost process is exacerbated in that that CO2 is known to convert to acid (e.g., carbonic acid) when in the presence of water (found in any kind of geological formation), resulting in higher capital equipment costs.

On the other hand, nature-based technology solutions may be of use, since realization may be affordable, secure, and immediate. Many governments are taking steps to reduce CO2 through national policies that include the introduction of emissions trading programs, voluntary programs, carbon or energy taxes and incentives, and regulations and standards on energy efficiency and emissions.

Through policy and legislation, organizations are increasingly required to measure, track and accurately report CO2 emissions. The creation of the emissions trading programs has created a market in which companies may trade in units called “carbon credits.” Thus, companies can create an additional source of profits by reducing CO2 emissions. But also, a company can reduce its CO2 emissions, gain carbon credits as a result of the emissions reduction, and then sell those credits in the open market for a profit.

Carbon offsets are also possible (also referred to as carbon reduction programs), which recognizes emission reductions in another form. That is, a carbon offset represents a reduction in emissions of CO2 where one party that produces emissions can ‘offset’ its emissions by purchasing carbon offsets from another party which has achieved carbon reductions through certain activities.

The sale of carbon offsets has a downstream effect to fund additional activities that reduce emissions, such as renewable energy projects (e.g., wind farms, hydroelectric dams, biomass energy) and energy efficiency projects. Moreover, the offset market presents a potential for an additional revenue stream, which may be advantageous in industries where no additional resource investment is needed. For example, where a farmer is already farming, the farmer may be able to realize revenue in the form of a carbon offset by just performing what is already performed-farming with increased focus and benefits of nature-based carbon sequestration. As one of skill would appreciate, the presence of biomass results in a natural sequester of CO2 (or carbon constituent) into the ground top and deep soil.

However, the amount of sequester or offset of carbon is not readily known or accurately measured. And when reporting sequestered carbon amounts, this cannot be random or based on a whim, as reporting is subject to protocol, oversight, and scrutiny; instead, data supported by reliable evidence is now of significant importance.

Accordingly, there is a need in the art to estimate an amount of sequestered carbon with precision, reliability, and repeatability, as the greater the validation the greater the likelihood of success on the credit offset market.

The industry needs a reliable, low-cost diagnostic method that can be used for assessing or estimating sequestered carbon amounts, and other soil performance parameters, as it may be the case that improved soil sequester performance is possible through sample analysis and data tracking. There is a need for a method of estimating sequestered carbon amounts that is versatile, affordable, highly accurate, non-radioactive, and expeditious. What is needed is a new and improved way of forming and using a fast, cost-favorable, effective, and reliable way of evaluating soil performance, which may be repeated as desired at periodic intervals.

### SUMMARY

Embodiments of the disclosure pertain to a method of estimating (determining, calculating, etc.) an amount of a (sequestered) carbon-based constituent in a volume of soil. The method may include taking a core soil sample, such as from a sample volume of soil. The sample volume of soil may be associated with a root system. As such, the sample volume of soil may be proximate, at, and/or below the root system. The root system may be part of or associated with carbon soil accumulation from a biomass. Examples of suitable biomass may include any of plants, trees, crops, and combinations thereof, while other forms of biomass are possible.

The method may include taking the core sample at a range of depth which may extend from an earthen surface. The method may include another step, such as testing the core sample to provide a data set. The method may include using the data set (or based thereon) to provide an estimation of the amount of the carbon-based constituent present in the volume of soil.

The carbon-based constituent may be or may include carbon dioxide (CO2). For example, CO2 may include or refer to organic carbon, inorganic carbon, and combinations thereof.

In aspects, the biomass may be associated with a peripheral activity, such as at least one of an agricultural operation, an oilfield operation, a livestock operation, and combinations thereof. In other aspects, the range of depth may include a depth of at least 10 feet below, but no more than 25 feet below, the earthen surface. The range may be any range between 10 feet to 25 feet, while other depths are possible.

In other aspects, the testing step may include one or more of any of the following: high-sensitivity infrared gas analysis, infrared gas analysis, soil moisture content analysis, nano laser diffraction analysis, x-ray diffraction, sub-atomic energy-dispersive x-ray spectroscopy, soil proctor compaction, wet and dry density assessment, soil moisture content analysis, and combinations thereof. The testing step may include heating an at least a portion of the core sample to a temperature in a range of about 1000 degrees F. to about 1500 degrees F. This step may include mixing a catalyst with the at least a portion of the core sample.

The method may have a sampling and testing frequency associated with it. For example, the method may include performing the taking the core soil sample and testing the core soil sample at least annually every year for a period of time. The period of time may be, for example, from at least one year to no more than fifty years. The period of time may be any number of years, including within the range of one year to fifty years.

The method may include adjusting a property or a parameter of the volume of soil based on the data set. For example, the property or the parameter may include one or more of: soil composition, particle size distribution, an amount or type of microbes or microbic material, phase composition, crystallinity, pH, and combinations thereof.

It may be the case that a plurality of soil samples may be taken, such as to provide a density of measurements at a predetermined interval range of the volume of soil. For example, a first tract of land may have a first soil sample taken. Then, a second or another tract of land may have a respective soil sample taken.

The method may include taking a plurality of samples, such as at different locations. How many samples to take or where to take may be based on a general understanding of the soil, such as one or more: of: soil properties, soil characteristics, soil heterogeneity, and combinations thereof.

The method may include using a proxy for estimating or supplementing the estimating. For example, the method may include using a form of a lateral measurement within the range of the depth to supplement estimating the amount of the carbon-based constituent. The form of lateral measurement may be or include one or more of: electric resistivity test, radar, ground penetrating radar, seismic analysis, satellite, and combinations thereof.

The method may include taking a core sample of soil, and testing the sample in order to analyze the soil in order to provide a set of data. Then, integrating the set of data with other formation data in order to determine a parameter associated with performance of the soil.

In aspects, the testing the sample step may include using a fluorescence response-based analysis. For example, the fluorescence response-based analysis may include use of EDXRF.

These and other embodiments, features and advantages will be apparent in the following detailed description and drawings.

### BRIEF DESCRIPTION OF THE DRAWINGS

A full understanding of embodiments disclosed herein is obtained from the detailed description of the disclosure presented herein below, and the accompanying drawings, which are given by way of illustration only and are not intended to be limitative of the present embodiments, and wherein:

FIG. 1A shows a system using tract of land with biomass for sequestering a carbon-based constituent according to embodiments of the disclosure;

FIG. 1B shows a side sectional subterranean view of a formation for the tract of FIG. 1A showing a root system according to embodiments of the disclosure;

FIG. 1C shows a simplified block diagram of an analytical unit used to test a sample of material (such as soil) according to embodiments of the disclosure;

FIG. 2A shows a downward view of a grid of tracts of land with biomass according to embodiments of the disclosure;

FIG. 2B shows a side sectional subterranean view of a formation whereby a form of lateral measurement is used between sampling points according to embodiments of the disclosure;

FIG. 3A shows a simplified flow chart showing a method of estimating an amount of a sequestered carbon-based constituent in a volume of soil,

FIG. 3B shows a simplified flow chart showing additional information for the method of FIG. 3A according to embodiments of the disclosure; and

FIG. 3C shows another simplified flow chart showing additional information for the method of FIGS. 3A and 3B according to embodiments of the disclosure.

### DETAILED DESCRIPTION

Regardless of whether presently claimed herein or in another application related to or from this application, herein disclosed are novel apparatuses, units, systems, and methods that pertain to carbon (or ‘CO2’) sequestration volume validation and duration of storage via advanced soil sampling by deep and shallow in-depth measurements, and using data for lateral propagation, details of which are described herein.

Embodiments of the present disclosure are described in detail with reference to the accompanying Figures. In the following discussion and in the claims, the terms “including” and “comprising” are used in an open-ended fashion, such as to mean, for example, “including, but not limited to . . . ”. While the disclosure may be described with reference to relevant apparatuses, systems, and methods, it should be understood that the disclosure is not limited to the specific embodiments shown or described. Rather, one skilled in the art will appreciate that a variety of configurations may be implemented in accordance with embodiments herein.

Although not necessary, like elements in the various figures may be denoted by like reference numerals for consistency and ease of understanding. Numerous specific details are set forth in order to provide a more thorough understanding of the disclosure; however, it will be apparent to one of ordinary skill in the art that the embodiments disclosed herein may be practiced without these specific details. In other instances, well-known features have not been described in detail to avoid unnecessarily complicating the description. Directional terms, such as “above,” “below,” “upper,” “lower,” “front,” “back,” etc., are used for convenience and to refer to general direction and/or orientation, and are only intended for illustrative purposes only, and not to limit the disclosure.

Connection(s), couplings, or other forms of contact between parts, components, and so forth may include conventional items, such as lubricant, additional sealing materials, such as a gasket between flanges, PTFE between threads, and the like. The make and manufacture of any particular component, subcomponent, etc., may be as would be apparent to one of skill in the art, such as molding, forming, press extrusion, machining, or additive manufacturing. Embodiments of the disclosure provide for one or more components to be new, used, and/or retrofitted to existing machines and systems.

Various equipment may be in fluid communication directly or indirectly with other equipment. Fluid communication may occur via one or more transfer lines and respective connectors, couplings, valving, piping, and so forth. Fluid movers, such as pumps, may be utilized as would be apparent to one of skill in the art.

Numerical ranges in this disclosure may be approximate, and thus may include values outside of the range unless otherwise indicated. Numerical ranges include all values from and including the expressed lower and the upper values, in increments of smaller units. As an example, if a compositional, physical or other property, such as, for example, molecular weight, viscosity, melt index, etc., is from 100 to 1,000. It is intended that all individual values, such as 100, 101, 102, etc., and sub ranges, such as 100 to 144, 155 to 170, 197 to 200, etc., are expressly enumerated. It is intended that decimals or fractions thereof be included. For ranges containing values which are less than one or containing fractional numbers greater than one (e.g., 1.1, 1.5, etc.), smaller units may be considered to be 0.0001, 0.001, 0.01, 0.1, etc. as appropriate. These are only examples of what is specifically intended, and all possible combinations of numerical values between the lowest value and the highest value enumerated, are to be considered to be expressly stated in this disclosure. Numerical ranges are provided within this disclosure for, among other things, the relative amount of reactants, surfactants, catalysts, etc. by itself or in a mixture or mass, and various temperature and other process parameters.

### Terms

The term “connected” as used herein may refer to a connection between a respective component (or subcomponent) and another component (or another subcomponent), which can be fixed, movable, direct, indirect, and analogous to engaged, coupled, disposed, etc., and can be by screw, nut/bolt, weld, and so forth. Any use of any form of the terms “connect”, “engage”, “couple”, “attach”, “mount”, etc. or any other term describing an interaction between elements is not meant to limit the interaction to direct interaction between the elements and may also include indirect interaction between the elements described.

The term “fluid” as used herein may refer to a liquid, gas, slurry, single phase, multi-phase, pure, impure, etc. and is not limited to any particular type of fluid such as hydrocarbons.

The term “utility fluid” as used herein may refer to a fluid used in connection with any fluid disposed into a wellbore (akin to an injection fluid). The utility fluid may be pressurized, and may be used to carry an additive into the wellbore. ‘Utility fluid’ may also be referred to and interchangeable with ‘service fluid’ or comparable.

The term “fluid connection”, “fluid communication,” “fluidly communicable,” and the like, as used herein may refer to two or more components, systems, etc. being coupled whereby fluid from one may flow or otherwise be transferrable to the other. The coupling may be direct, indirect, selective, alternative, and so forth. For example, valves, flow meters, pumps, mixing tanks, holding tanks, tubulars, separation systems, and the like may be disposed between two or more components that are in fluid communication.

The term “pipe”, “conduit”, “line”, “tubular”, or the like as used herein may refer to any fluid transmission means, and may be tubular in nature.

The term “sample” or “core sample” (or the like) may refer to taking a sample of a volume of material, such as soil. Taking the sample may include the use of all necessary equipment (including auxiliary), such as that needed for coring while drilling or rotary sidewall coring.

The term “composition” or “composition of matter” as used herein may refer to one or more ingredients, components, constituents, etc. that make up a material (or material of construction). Composition may refer to a flow stream of one or more chemical components.

The term “chemical” as used herein may analogously mean or be interchangeable to material, chemical material, ingredient, component, chemical component, element, substance, compound, chemical compound, molecule(s), constituent, and so forth and vice versa. Any ‘chemical’ discussed in the present disclosure need not refer to a 100% pure chemical. For example, although ‘water’ may be thought of as H2O, one of skill would appreciate various ions, salts, minerals, impurities, and other substances (including at the ppb level) may be present in ‘water’. A chemical may include all isomeric forms and vice versa (for example, “hexane”, includes all isomers of hexane individually or collectively).

The term “carbon-based constituent” as used herein may refer to any component having a carbon element in some form (e.g., elemental, isomer, ion, molecule, compound, etc.). CO and CO2 are an example of a carbon-based constituent. The total value of a carbon-based constituent may be a summation of all carbon present, which may be expressed in concentration or percentage.

The term “water” as used herein may refer to a pure, substantially pure, and impure water-based stream, and may include wastewater, process water, fresh water, seawater, produced water, slop water, treated variations thereof, mixes thereof, etc., and may further include impurities, dissolved solids, ions, salts, minerals, and so forth.

The term “impurity” as used herein may refer to an undesired component, contaminant, etc. of a composition. For example, a mineral or an organic compound may be an impurity of a water stream.

The term “process” or “processing” as used herein may refer to some kind of active step or action, such as man-made or by machine, imparted on a material (such as a solid or fluid. For example, a sample of soil may be received into a device (such as a sample analyzer) and upon processing, may leave as a ‘processed material’. ‘Processed’ is not meant be limited, as this may include reference to transferred, treated, tested, measured, mixed, sensed, separated, combinations, etc. in whatever manner may be desired or applicable for embodiments herein. It is noted that while various steps or operations of any embodiment herein may be described in a sequential manner, such steps or operations may be operated in batch or continuous fashion. Although not necessary, system and process may have a comparable meaning.

The term “EDXRF” (Non-destructive Energy Dispersive X-Ray Fluorescence) as used herein may refer to a type of spectroscopy process (and may thus include use of a spectrometer) where a sample of material (such as a portion of produced fluid) is ‘excited’ in order to collect emitted fluorescence radiation, which may then be evaluated for different energies of the characteristic radiation from each of the different constituents (or elements) in the sample. The EDXRF process may be referred to as a fluorescence response-based analytical process.

EDXRF may be considered a non-destructive analytical technique used to determine the elemental composition of materials. EDXRF analyzers determine the elemental composition of a sample by measuring the fluorescent (or secondary detectable energy) X-ray emitted from a sample when it is excited by a primary X-ray source. EDXRF is designed to analyze groups of elements simultaneously to determine those elements presence in the sample and their relative concentrations—in other words, the elemental composition of the sample.

Each of the elements present in a sample produces a unique set of characteristic X-rays that is a “fingerprint” for that specific element. X-rays have a very short wavelength, which corresponds to very high energy. All atoms have several electron orbitals (K shell, L shell, M shell, for example). When X-ray energy causes electrons to transfer in and out of these shell levels, X-ray fluorescence peaks with varying intensities are created and will be present in the spectrum. The peak energy identifies the element, and the peak height or intensity is indicative of its concentration.

The term “XRD” may refer to X-ray diffraction, which is a technique for analyzing the atomic or molecular structure of materials. It is non-destructive, and works most effectively with materials that are wholly, or part, crystalline. The technique is often known as x-ray powder diffraction because the material being analyzed typically is a finely ground down to a uniform state. Diffraction is when light bends slightly as it passes around the edge of an object or encounters an obstacle or aperture. The degree to which it occurs depends on the relative size of a wavelength compared to the dimensions of the obstacle or aperture it encounters.

All diffraction methods start with the emission of x-rays from a cathode tube or rotating target, which is then focused at a sample. By collecting the diffracted x-rays, the sample's structure can be analyzed. This is possible because each mineral has a unique set of d-spacings. D-spacings are the distances between planes of atoms, which cause diffraction peaks.

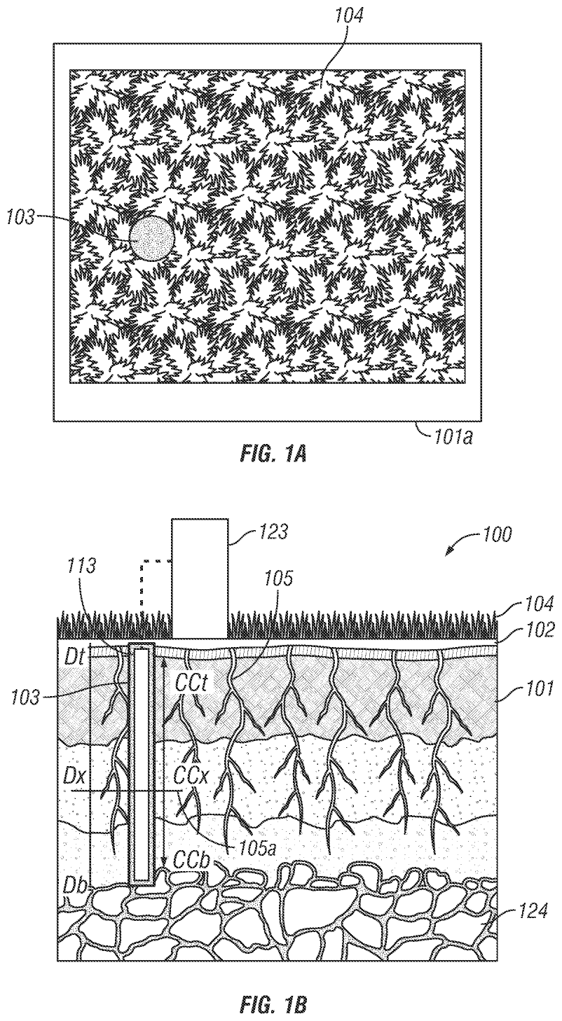

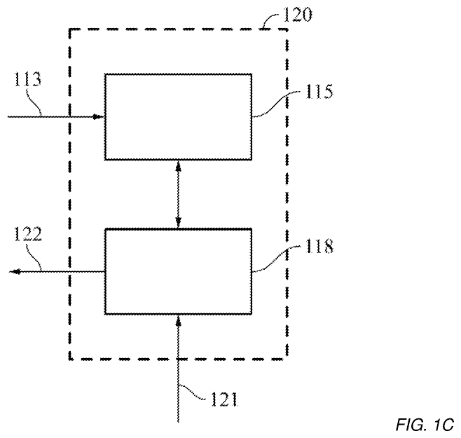

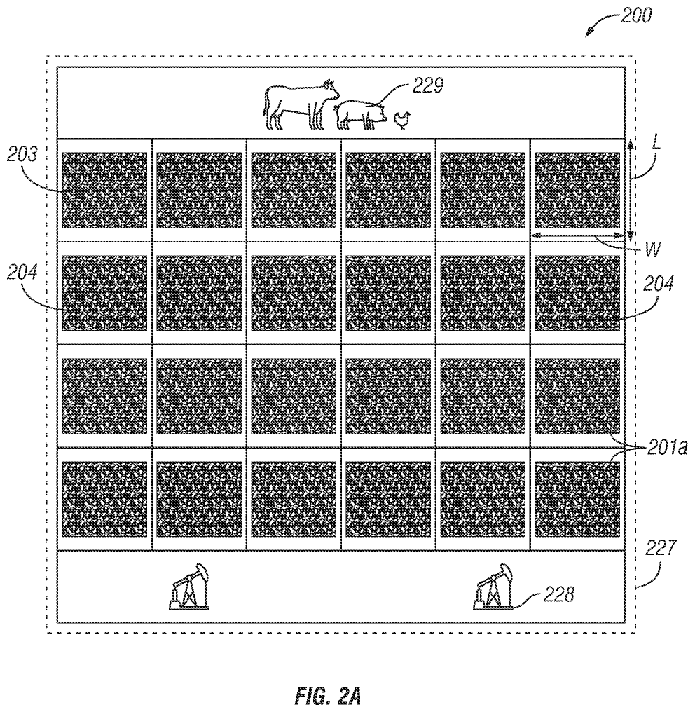

Referring now to FIGS. 1A, 1B, and 1C together, a downward view of a system using tract of land with biomass for sequestering a carbon-based constituent, a side sectional subterranean view of a formation for the tract of FIG. 1A showing a root system, and a simplified block diagram of an analytical unit used to test a sample of material (such as soil), respectively, according to embodiments disclosed herein, are shown.

The system 100 may include one or more components (or subcomponents) coupled with new, existing, or retrofitted equipment. System 100 may include one or more units that are skid mounted or may be a collection of skid units.

FIGS. 1A-1C are meant to show in a simplistic manner embodiments herein, and may not be to scale. The system 100 may have various valves, flanges, pipes, pumps, utilities, monitors, sensors, controllers, flow meters, safety devices, etc., for accommodating sufficient universal coupling between system components and any applicable source of a material to be processed, any resultant product material to be discharged or transferred therefrom, and anything in between.

The system 100 may include a surface (e.g., earthen surface) 102 having a biomass 104 disposed or otherwise growing thereon. The biomass 104 may be a typical form of material or substance known to live on and grow from the ground. For example, the biomass 104 may be grass, weeds, trees, bushes, or any kind of (agricultural) crop (e.g., wheat, corn, etc.). Still other examples may include waste or other visible materials, but also less visible or microscopic materials, such as algae. The biomass 104 may be that which produces oxygen (O2) to the surrounding atmosphere via a photosynthesis reaction.

Through photosynthesis, the biomass 104 may absorb CO2 from the atmosphere, and then use water and sunlight to turn the carbon into leaves, stems, seeds and root system 105. During a normal life cycle, the biomass 104 may return some CO2 to the atmosphere, but other amounts of CO2 may aggregate and absorb into a subterranean formation 101 around the root system 105.

The biomass 104 may not be limited to a single type of biomass, and thus may be combinations of different forms, such as grass and trees referred to collectively. The biomass 104 may have or be associated with the root or root system 105. The root system 105 may include roots that are various and interwoven. Thus, the root system 105 may include a root(s) of different types of biomass 104. The root system 105 may include a part of the biomass 104 that attaches and grows in the subterranean formation (ground) 101 underneath the surface 102. The root system 105 may convey water and other nourishment to the biomass, such as via plentiful branches and fibers 105a.

As a result of the ability of the formation 101 to sequester and store CO2 (or any carbon-based constituent “CC”), there may be a range of CC concentration from CCₜ to CC_{b}. The range of CC concentration may vary, for example, at a constant depth but varied on a lateral. The CC concentration may also vary along a depth (or range of depth), such as from a top depth Dt (e.g., a depth of soil adjacent or just at the surface 102) to a bottom depth D_{b}. The concentration of carbon constituent at any particular depth Dt may be indicated as CCₓ. The concentration of CC may vary and/or be non-linear. For example, at the top depth Dt the concentration CCₜ may be higher than the concentration CCₓ at the depth Dₓ. At the same time, the bottom concentration CC_{b} may be a value in-between, higher, or lower. Although not discussed in detail, the CC concentration may also vary on a lateral or horizontal due to additional heterogeneity associated with the formation 101.

The best way to understand the amount of CC within the formation 101 may be via taking a soil or core sample 113. This may be done, for example, by having surface sample equipment 123. The equipment may include sufficient equipment for drilling a wellbore (e.g., open bore) 103 in the formation 101 to any desired (bottom) depth D_{b}. Either during drilling or after, the core sample 113 may be obtained.

Although not limited, the depth of the wellbore 103 may extend all the way from the surface 102 to a subterranean barrier (e.g., caprock) 124. In the embodiments shown here, the wellbore 103 may have a generally vertical portion; however, as one of skill would appreciate the shape of the bore 103 is not limited, and could include horizontal, vertical, slant, curved, directional, and/or other well geometries, and combinations.

Once the wellbore 103 is drilled sufficiently, the equipment 123 may be used to take a column or sample of the soil 113. The sample 113 may be any amount of soil as desired, and may be done up to any depth Dₓ in the formation 101. Taking and testing of the sample 113 may be repeated at predetermined or desired intervals, such as once a year, and repeated for any number of years after, such as from ten to fifty (or more).

The sample 113 may now be tested via test unit 120. The test unit 120 may include analysis equipment 115, which may be in operable communication with computing system 118. The computing system 118 may be configured for use in using analytical data associated with use of the test equipment 115. The test equipment 115 may provide a fluorescence response-based process, such as EDXRF and XRD.

The computing system 118 may be useful to further analyze data and other information in order to provide an indication related to performance of the formation 101 in terms of sequestration.

The computing system 118 may have artificial intelligence (A.I.) based flow diagnostics. The computing system 118 may access input data 121, which may be related to other aspects of the formation 101, such as soil moisture information, composition, and the like. The computing system 118 may include programs, scripts, and/or other types of computer instructions that generate output data 122, which may be based on the input data 121. The output data 122 may include an estimation or calculation of an amount of CC present within the sample 113, and extrapolated to an amount of CC within an entire volume of soil associated with the tract 101a.

The output data 122 may be used to validate amounts of the CC present within the tract 101a, thereby establishing and confirming an amount of carbon offsets available for sale or trade. The output data 122 may be used to determine a soil parameter that may be adjusted in order to increase yield and soil performance, thereby improving (increasing) amounts of sequestered CC.

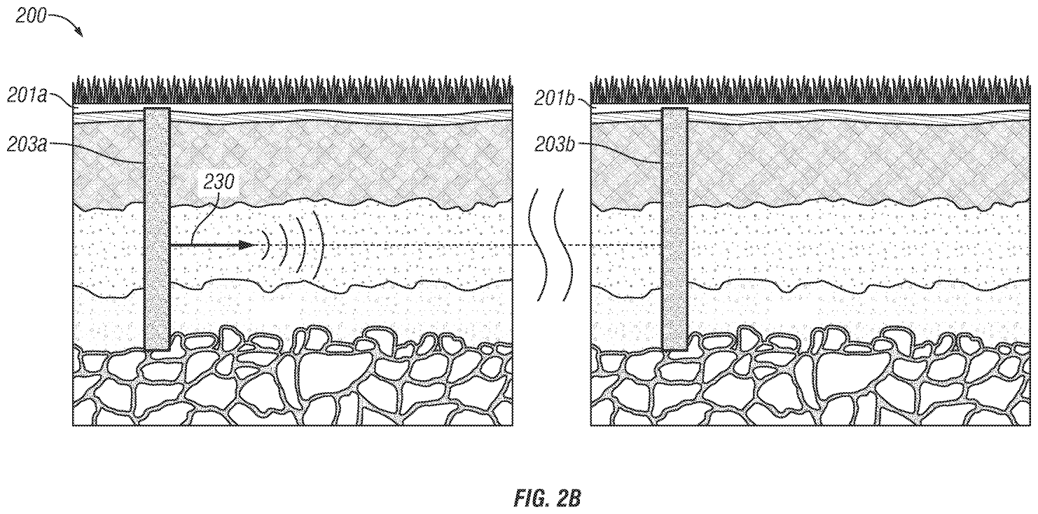

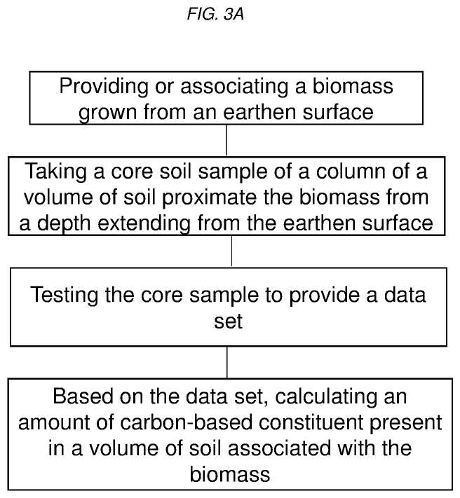

Referring now to FIGS. 2A and 2B together, a downward view of a grid of tracts of land with biomass and a side sectional subterranean view of a formation whereby a form of lateral measurement is used between sampling points, respectively, according to embodiments disclosed herein, are shown. The system 200 may be like that of system 100, with the understanding that a first land tract (101a) may be part of a grid arrangement that includes a plurality of land tracts 201a. The plurality of land tracts 201a may be part of an overall larger land tract 227 that is broken down into a grid system (such as each tract 201a having a determinable perimeter and surface area [L×W]). One of skill would appreciate the tracts 201a need not be uniform or symmetrical and may take any shape or size.

Any of the tracts 201a may have a biomass 204 and root system (105) according to embodiments herein (see previously described 104, 105). The estimation or calculation of sequestered carbon for each tract 201a may be done as described for tract 101a.

Of interest, the system 200 may be associated with an already existing operation, such as an oilfield operation 228 or a livestock/farm operation 229. As such, the system 200 may be used to realize profit and income from not just the biomass 204 or operations 228, 229, but also from validated carbon offsets.

Below a respective land tract 201a, the soil and formation may not be (and likely is not) homogenous, and thus any estimation of carbon constituent (CC) may be prone to inaccuracy. Accuracy may be improved by taking samples at much closer distances. In the alternative, the estimation may be improved or supplemented by way of a proxy measurement.

As shown in FIG. 2B, even though a respective sample may be taken via first bore 203a, the formation may not be homogenous along a lateral (such as lateral distance DL). The lateral distance DL may be that between where a first sample is taken (bore 203a) to where another sample is taken (from another bore 203b). The other bore 203b may be the sample point for another or second tract 201b.

The proxy measurement may be in a form of a lateral measurement (which may be at any depth, and could be done at different depths). The form of the lateral measurement, may be, for example, electric resistivity test, radar, ground penetrating radar, seismic analysis, satellite, and other comparable test or measurements, and combinations thereof.

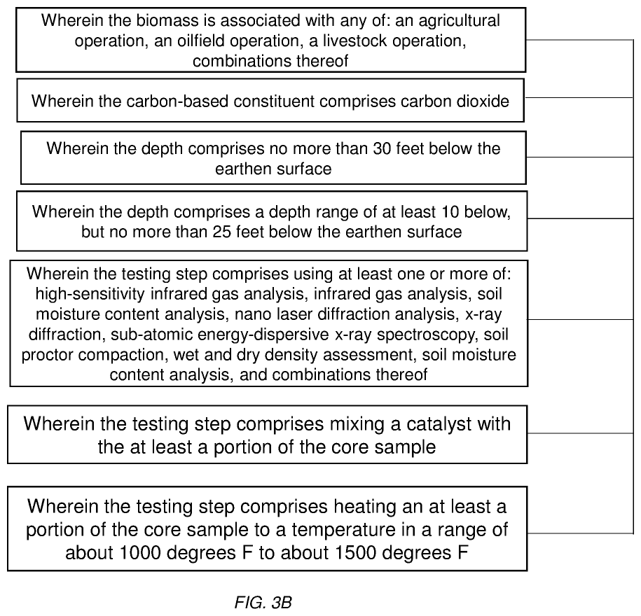

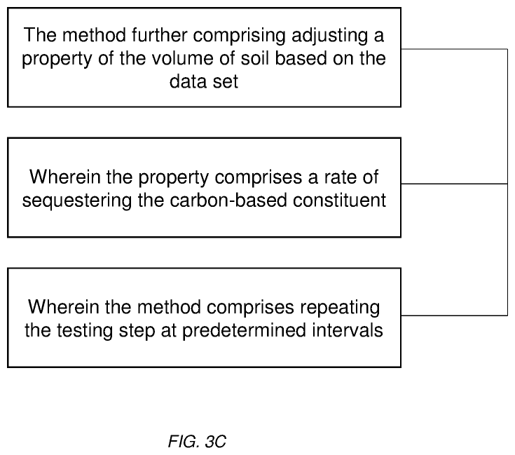

Referring now to FIGS. 3A, 3B, and 3C, a simplified flow chart showing a method of estimating an amount of a sequestered carbon-based constituent in a volume of soil, a simplified flow chart showing additional information for the method of FIG. 3A, and another simplified flow chart showing additional information for the method of FIGS. 3A and 3B, respectively, in accordance with embodiments disclosed herein, are shown.

FIGS. 3A-3C together show diagrams related to a method (or process, system, etc.), including one or more steps thereof, for estimating an amount of a sequestered carbon-based constituent in a volume of soil.

The method may include providing (using, growing, associating, etc.) a biomass, which may grow or be grown from an earthen surface of a land tract.

The method may include taking a (core) soil sample of a volume of soil (which may be a column) proximate a root system of the biomass. The soil sample may be taken at a depth (or range of depth if taken as a column or comparable) extending from the earthen surface.

The depth or range of depth may be any particular value. For example, the depth may include a depth range of at least 10 below, but no more than 25 feet below the earthen surface. The depth may extend to a bottom or barrier, which may be caprock or other type of impermeable geological formation. The depth may be no more than 30 feet below the earthen surface.

The method may include testing the core sample to provide a data set. Based on the data set, the method may include estimating or calculating an amount of carbon-based constituent present in a volume of soil associated with the root system (which may be of the biomass). The carbon-based constituent may be or include carbon dioxide, or other carbon variants.

The biomass may be associated with any of: an agricultural operation, an oilfield operation, a livestock operation, combinations thereof. The agricultural operation may be, for example, farming or growing/harvesting crops. Other agrarian operations may be possible, such as a passive tree farm.

The testing step may include using at least one or more of: high-sensitivity infrared gas analysis, infrared gas analysis, soil moisture content analysis, nano laser diffraction analysis, x-ray diffraction, sub-atomic energy-dispersive x-ray spectroscopy, soil proctor compaction, wet and dry density assessment, soil moisture content analysis, and combinations thereof.

The testing step may include mixing a catalyst with the at least a portion of the core sample. Whether a catalyst is used and/or what type of catalyst may depend on what type of testing and analysis is used. The testing step may include heating an at least a portion of the core sample to a temperature in a range of about 1000 degrees F. to about 1500 degrees F.

Embodiments of the method may facilitate assessing whether the volume of soil associated with the root system may be changed in order to benefit yield and overall amount of carbon sequestration. As such, the method may include the step of adjusting a property of the volume of soil based on the data set. For example, adding a fertilizer into the soil may improve yield. As such, the property adjusted may include a rate or an ability (characteristic) of sequestering the carbon-based constituent.

The method may include repeating the testing step at predetermined intervals, and may also be done in other land tracts proximate to the location of the root system.

### Example

Embodiments herein provide for a method of calculating or estimating an amount of carbon constituent present in a volume of soil. This may occur via carbon verification and soil analysis technology that may utilize advanced sub-atomic and nano-particle analytical expertise and capabilities to enable farmers, ranchers, and other landowners to maximize value in the flourishing carbon credit marketplace. The comprehensive core sampling, ultra-high-resolution carbon measurements, and subsurface soil diagnostic initiative has quantified nature-based carbon sequestration levels higher/acre (25X+) than those recorded using conventional reporting methodologies.

The precise quantification of appreciably higher carbon captured volumes advance carbon offset initiatives by increasing available carbon credits/acre landowners can sell in the carbon credit market and to oil and gas operators and other companies. The companies, in turn, can use the credits to offset emissions generated by their operations to help meet net zero carbon objectives and other critical environmental commitments. Increasing quantifiable and high-quality carbon credits gives operators a secure and low-cost mechanism to offset emissions, while affording landowners an attractive incremental revenue stream.

Carbon verification and soil analysis of embodiments herein goes well beyond shallow soil sampling and testing methodologies. For example, embodiments herein may include soil samples from up to 10 ft or deeper, well below the root systems of most crops and trees, which are natural sequester magnets for atmospheric CO2. Testing results from multiple agricultural-related projects collocated with associated activities have verified carbon capture quantities increasing from the 3 tons/acre previously recorded to more than 81 tons/acre.

Through comprehensive testing protocol of the present disclosure, carbon verification and soil analysis may be thoroughly quantified. Embodiments herein may result in additional income to farmers, ranchers and landowners, while helping public and private companies achieve their carbon neutral and ESG objectives.

The sample may then be tested via a fluorescence response-based process, such as EDXRF and XRD. Such analytical techniques may be used to determine the elemental composition and crystallinity of the samples.

EDXRF is designed to analyze groups of elements simultaneously to determine those elements presence in the sample and their relative concentrations—in other words, the elemental composition of the sample. Each of the elements present in a sample produces a unique set of characteristic X-rays that is a “fingerprint” for that specific element. X-rays have a very short wavelength, which corresponds to very high energy.

Due to sub-atomic accuracy of both detection methods, it is possible to precisely determine the elemental composition, crystallographic structure, and the various combinations of hyperfine interactions in the samples, which enables very accurate identification of the carbon-based constituents on the sub-atomic or quantum level.

Laboratory analysis that may include or incorporate advanced computational methods and proprietary diagnostics capabilities for each stage or target formation provides accurate, calibrated, actionable and cost-effective production diagnostics results. This enables operators to reduce operational cost and increase the production in oil and gas wells.

Embodiments herein may produce and achieve an extensive and long-term datasets. This information may be used together with advanced computational methods using artificial intelligence (A.I.) coupled with artificial neural network may provide precise estimation and validation of amounts of sequestered carbon (CC).

The method may include taking a sample of soil, and then testing the sample in order to analyze the soil to provide a set of data. The method may include integrating (or otherwise analyzing, comparing, etc.) the set of data with other data in order to determine a parameter (or property, characteristic, etc.) associated with performance of the soil. The set of data may be used to take a response to improve the sequestration performance of the soil.

The testing the sample step may include using a fluorescence response-based analysis. In aspects, the fluorescence response-based analysis may include use of EDXRF. In aspects, the fluorescence response-based analysis many include use of XDR.

### Example Testing Methods

1. Soil Proctor Compaction Test: The proctor compaction test is a laboratory method of experimentally determining the optimal moisture content at which a given soil type will become most dense and achieve its maximum dry density. The soil is usually compacted into the mold in a certain number of equal layers, each receiving several blows from a standard weighted hammer at a specified height. This process is then repeated for various moisture contents, and the dry densities are determined for each. The graphical relationship of the dry density to the moisture content is then plotted to establish the compaction curve. The maximum dry density is finally obtained from the peak point of the compaction curve and its corresponding moisture content, also known as the optimal moisture content.

2. Particle Size Distribution (PSD) and Surface Area Analysis by Laser Diffraction: The soil particle size distribution and surface area analysis are performed to determine the percentage of each size and surface area of grain particles that is contained within a soil sample, and the results of the test can be used to produce the grain size distribution and surface area curves. This information is used to classify the soil, characterize the soil lithology, and predict its behavior for a nature-based carbon sequestration process. The technique employed is a nano laser diffraction analysis to measure the particle size, particle size distribution, and particle surface area of each soil sample. It does this by measuring the intensity of light scattered as a laser beam passes through a dispersed particulate soil sample. Large particles scatter light at small angles relative to the laser beam, and small particles scatter light at large angles. The angular scattering intensity data is then analyzed to calculate the size and surface area of the soil particles that created the scattering pattern using the Mie method of light scattering.

3. Soil Compound and Mineral Analysis: The soil compound and mineral analysis is performed using advanced X-ray diffraction (XRD) analysis. The XRD is a versatile, non-destructive analytical technique used to analyze physical properties such as phase composition, crystal structure, and orientation of soil particles. The XRD is the technique heavily relied on in the most accurate soil compound, mineralogical, and lithology analyses. X-ray diffraction is a sub-atomic technique that provides detailed information about the atomic structure of crystalline substances in each soil sample. It is a very powerful tool in the identification of compounds and minerals in rock and soil samples.

An XRD instrument contains three main items: a high-energy X-ray source, a sample holder, and a highly sensitive XRD detector. The X-rays produced by the source illuminate the sample and its structure. It is then diffracted by the sample phase and enters the detector. By moving the tube or sample and detector to change the diffraction angle (20, the angle between the incident and diffracted beams), the intensity is measured, and diffraction data are recorded. Depending on the geometry of the diffractometer and the type of soil sample, the angle between the incident beam and the sample can be either fixed or variable and is usually paired with the diffracted beam angle.

4. Soil Moisture Content Analysis: The moisture content of a soil sample is described as the ratio of the mass of water held in the soil to the dry soil. The mass of water is determined by the difference before and after drying the soil. Moisture content is how much water is in a product. It influences the physical properties of a soil, including weight, density, viscosity, conductivity, and others. It is generally determined by weight loss upon drying. The mass of moist soil consists of the mass of the dry soil particles plus the mass of the water within the soil. The dry mass of the soil particles is fixed, whereas the amount of water within moist soil can vary. Therefore, moisture content is calculated on a dry basis rather than a total mass basis to ensure consistency. The moisture content of soil is described as the ratio of the mass of water held in the soil to the dry soil. The mass of water is determined by the difference before and after drying the soil. Although the measurement is simple, it is important to determine soil moisture content to better understand soil characteristics.

5. Soil Density Analysis: A soil density test is a type of material test to determine the density of compacted soil, rock, or other materials in a lab setting. This is an important test for soil characterization, where a particular density assessment is required for soil carbon sequestration analysis. There are several techniques available for soil density testing. The traditional method involves taking a core sample in a tube and weighing the sample to determine how much soil, sand, or other fine material fits in each area with the current level of compaction. The materials can be weighed dry and wet to provide additional information about their density. Dry density is calculated by dividing the weight of the wet soil by its water content in percent.

6. Soil Carbon and Organic Content Analysis: Soil carbon and organic content analysis, which is a major component of soil organic matter, is considered perhaps the most important measurements of actual soil carbon sequestration verification and quantification, soil quality, and productivity. A common and most accurate method is the dry soil combustion method, which measures the total carbon (TC), inorganic carbon (IC), and total soil organic carbon (TOC/SOC). The combustion method achieves total combustion of soil samples by heating them to 680° C. in an oxygen-rich environment inside combustion tubes filled with a platinum catalyst. The carbon dioxide generated by oxidation is detected using an infrared gas analyzer (NDIR). The high-sensitivity NDIR measurement achieves high detection sensitivity, with a detection limit of 4 μg/L, which is the highest level for the carbon detection and quantification method for soil sample analysis. The determination of the inorganic carbon can also be carried out in a separate furnace in the module. The inorganic carbon in a soil sample is measured by pre-mixing the soil sample in phosphoric acid, and the resulting CO2 is purged at 200° C. and measured using an infrared gas analyzer.

7. Soil Geochemical and Elemental Analysis: Geochemical analysis is the process that determines the chemical compounds that constitute soil samples rheology and composition. The building blocks of any soil sample are the chemical elements. These elements can be identified by their atomic number Z, which is the number of protons in the nucleus. An element can have more than one value for N, the number of neutrons in the nucleus. The sum of these is the mass number, which is roughly equal to the atomic mass.

These elements, which include Na, K, Si, Al, Ti, Mg, Ca, and many others, form silicates and other oxides in the soil samples. The sub-atomic energy-dispersive X-ray fluorescence (EDXRF) spectrometry is used for the soil samples geochemical and elemental analyses. EDXRF is a nondestructive, rapid, multielement, highly accurate, and environment-friendly analysis compared with other elemental detection methods. These benefits also apply to soil samples, for which the average detection limits for many elements of soil geochemistry and elemental importance are well below 1 ppm. The measurements are carried out directly on the sample itself, with little to no sample preparation or digestion. EDXRF is a non-destructive technique, and the sample can also be measured subsequently by other analytical techniques if required.

While embodiments of the disclosure have been shown and described, modifications thereof may be made by one skilled in the art without departing from the spirit and teachings of the disclosure. The embodiments described herein are exemplary only and are not intended to be limiting. Many variations and modifications of the embodiments disclosed herein are possible and are within the scope of the disclosure. Where numerical ranges or limitations are expressly stated, such express ranges or limitations should be understood to include iterative ranges or limitations of like magnitude falling within the expressly stated ranges or limitations. The use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the preferred embodiments of the present disclosure. The inclusion or discussion of a reference is not an admission that it is prior art to the present disclosure, especially any reference that may have a publication date after the priority date of this application. The disclosures of all patents, patent applications, and publications cited herein are hereby incorporated by reference, to the extent they provide background knowledge; or exemplary, procedural or other details supplementary to those set forth herein.

What is claimed is:

1. A method of estimating an amount of a sequestered carbon-based constituent in a volume of soil, the method comprising:
taking a core soil sample of a sample volume of soil proximate at and below a root system associated with carbon soil accumulation from a biomass, the core sample taken at a range of depth extending from an earthen surface;
testing the core sample to provide a data set; and
based on the data set, estimating the amount of the carbon-based constituent present in a volume of soil associated with the root system.

2. The method of estimating the amount of the carbon-based constituent of claim 1, wherein the biomass is associated with at least one of an agricultural operation, an oilfield operation, a livestock operation, and combinations thereof.

3. The method of estimating the amount of the carbon-based constituent of claim 1, wherein the carbon-based constituent comprises carbon dioxide.

4. The method of estimating the amount of the carbon-based constituent of claim 1, wherein the carbon-based constituent comprises organic carbon, inorganic carbon, and combinations thereof.

5. The method of estimating the amount of the carbon-based constituent of claim 1, wherein the range of depth comprises a depth of at least 10 feet below, but no more than 25 feet below, the earthen surface.

6. The method of estimating the amount of the carbon-based constituent of claim 1, wherein the testing step comprises using at least one of: high-sensitivity infrared gas analysis, surface area analysis by laser diffraction, infrared gas analysis, soil moisture content analysis, nano laser diffraction analysis, x-ray diffraction, geochemical analysis, sub-atomic energy-dispersive x-ray spectroscopy, soil proctor compaction, wet and dry density assessment, soil moisture content analysis, and combinations thereof.

7. The method of estimating the amount of the carbon-based constituent of claim 1, wherein the testing step comprises heating an at least a portion of the core sample to a temperature in a range of about 1000 degrees F. to about 1500 degrees F.

8. The method of estimating the amount of the carbon-based constituent of claim 7, wherein the testing step comprises mixing a catalyst with the at least a portion of the core sample.

9. The method of estimating the amount of the carbon-based constituent of claim 1, the method further comprising performing the taking the core soil sample and testing the core soil sample at least annually every year for a period of time of an at least one year to no more than fifty years.

10. The method of estimating the amount of the carbon-based constituent of claim 1, the method further comprising adjusting a property or a parameter of the volume of soil based on the data set.

11. The method of estimating the amount of the carbon-based constituent of claim 10, wherein either of the property or the parameter comprises one of: soil composition, particle size distribution, amount of microbes, phase composition, crystallinity, pH, and combinations thereof.

12. The method of estimating the amount of the carbon-based constituent of claim 1, wherein the biomass comprises an at least one of plants, trees, crops, and combinations thereof.

13. The method of estimating the amount of the carbon-based constituent of claim 1, wherein a plurality of soil samples are taken to provide a density of measurements at a predetermined interval range of the volume of soil.

14. The method of estimating the amount of the carbon-based constituent of claim 1, wherein a plurality of samples are taken at different locations based on at least one of: soil properties, soil characteristics, soil heterogeneity, and combinations thereof.

15. The method of estimating the amount of the carbon-based constituent of claim 1, the method further comprising using a form of a lateral measurement within the range of the depth to supplement estimating the amount of the carbon-based constituent.

16. The method of estimating the amount of the carbon-based constituent of claim 15, wherein the form of the lateral measurement comprises one of: electric resistivity test, radar, ground penetrating radar, seismic analysis, satellite, and combinations thereof.

17. A method of estimating an amount of a sequestered carbon-based constituent in a volume of soil, the method comprising:
taking a core soil sample of a sample volume of soil proximate at and below a root system associated with carbon soil accumulation from a biomass, the core sample taken at a range of depth extending from an earthen surface;
testing the core sample to provide a data set;
based on the data set, estimating the amount of the carbon-based constituent present in a volume of soil associated with the root system; and
adjusting a parameter of the volume of soil based on the data set,
wherein the biomass is associated with at least one of an agricultural operation, an oilfield operation, a livestock operation, and combinations thereof,
wherein the carbon-based constituent comprises carbon dioxide, and
wherein the range of depth comprises a depth of at least 10 feet below, but no more than 25 feet below, the earthen surface.

18. The method of estimating the amount of the carbon-based constituent of claim 17, wherein the testing step comprises using at least one of: high-sensitivity infrared gas analysis, infrared gas analysis, soil moisture content analysis, nano laser diffraction analysis, x-ray diffraction, sub-atomic energy-dispersive x-ray spectroscopy, soil proctor compaction, wet and dry density assessment, soil moisture content analysis, and combinations thereof.

19. The method of estimating the amount of the carbon-based constituent of claim 18, wherein the testing step comprises heating an at least a portion of the core sample to a temperature in a range of about 1000 degrees F. to about 1500 degrees F., and mixing a catalyst with the at least a portion of the core sample.

20. The method of estimating the amount of the carbon-based constituent of claim 19, the method further comprising using a form of a lateral measurement taken at a point within the range of the depth to supplement estimating the amount of the carbon-based constituent.
